(19) United States
(12) Patent Application Publication
(10) Pub. No.: US 2018/0244792 A1
(43) Pub. Date: Aug. 30, 2018

(54) ANTI-FIBROTIC EFFECT OF CD70

(71) Applicant: University of Pittsburgh - Of the Commonwealth System of Higher Education, Pittsburgh, PA (US)

(72) Inventor: Steven Duncan, Vestavia Hills, AL (US)

(73) Assignee: University of Pittsburgh - Of the Commonwealth System of Higher Education, Pittsburgh, PA (US)

(21) Appl. No.: 15/758,691

(22) PCT Filed: Sep. 9, 2016
(86) PCT No.: PCT/US2016/050954

§ 371 Date: Mar. 8, 2018

Related U.S. Application Data
(60) Provisional application No. 62/216,928, filed on Sep. 10, 2015.

(51) Int. Cl. C07K 16/28; A61K 38/17; A61P 11/00; A61P 17/00

(52) U.S. Cl. CPC C07K 16/2875; A61K 38/1793; A61K 2039/505; A61P 17/00; A61P 11/00

(57) ABSTRACT

Disclosed are methods for treating fibrosis in a subject using a CD70 agonist. In some examples, the agent is an antibody that binds CD70, an antigen biding fragments thereof, soluble CD27, or a small molecule. In some embodiments, the fibrosis is fibrosis of the skin and/or the lung. In some embodiments, the subject is administered a nucleic acid molecule encoding a CD70 agonist.

### CROSS REFERENCE TO RELATED APPLICATION

This application claims the benefit of U.S. Provisional Application No. 62/216,928, filed Sep. 10, 2015, which is incorporated by reference in its entirety.

### ACKNOWLEDGMENT OF GOVERNMENT SUPPORT

This invention was made with government support under grant nos. HL119960, HL107172 and HL084932 awarded by the National Institutes of Health. The government has certain rights in the invention.

### FIELD

This relates to the field of fibrosis, specifically to the use of CD70 agonists for the treatment of fibrosis.

### BACKGROUND

Excessive deposition of extra cellular matrix (ECM) components such as fibronectin (FN) and type I collagen (Collα1) by organ fibroblasts is defined as fibrosis. Organ fibrosis is the final common pathway for many diseases that result in end-stage organ failure. However, effective therapy for organ fibrosis is still unavailable (see, for example, Bjoraker et al., Am. J. Respir. Crit. Care. Med 2000;157: 199-203). Uncontrollable wound-healing responses, including acute and chronic inflammation, angiogenesis, activation of resident cells, and ECM remodeling, are thought to be involved in the pathogenesis of fibrosis (Wynn, J. Clin. Invest. 2007;117:524-29; Kalluri et al., Curr. Opin. Nephrol. Hypertens 2000;9:413-8). TGF-β is the prototype fibrotic cytokine that is increased in fibrotic organs and contributes to the development of fibrosis by stimulating the synthesis of ECM molecules, activating fibroblasts to α-smooth muscle actin (α-SMA)-expressing myofibroblasts, and downregulating matrix metalloproteinases (MMPs) (see, for example, Branton et al., Microbes Infect 1999;1:1349-65). Despite high expectations, a clinical trial of a monoclonal anti-TGF-β antibody in patients with early SSc failed to show any efficacy (Varga et al., Nature Reviews Rheumatology 2009; 5:200-6). Thus, a need remains for additional agents to treat fibrosis.

### SUMMARY

Methods are disclosed for treating fibrosis in a subject. These methods include administering to the subject a therapeutically effective amount of a CD70 agonist.

In some embodiments, the fibrosis is fibrosis of the skin or lung. In some non-limiting examples, the subject can have scleroderma, a keloid scar, a hypertrophic scar, pulmonary fibrosis such as idiopathic pulmonary fibrosis, morphea, Graft-Versus-Host Disease, or sub-epithelial fibrosis. The CD70 agonist can be administered systemically or locally.

In further embodiments, the CD70 agonist is a soluble (s)CD27, an antibody that specifically binds CD70, or an antigen binding fragment of the antibody. In further embodiments, the subject is administered a nucleic acid molecule encoding the sCD27, the antibody that specifically binds CD70, or the antigen binding fragment.

The foregoing and other objects, features, and advantages of the invention will become more apparent from the following detailed description, which proceeds with reference to the accompanying figures.

### BRIEF DESCRIPTION OF THE DRAWINGS

FIG. 1 shows that, in idiopathic pulmonary fibrosis, T cells express CD27 and CD28.

FIG. 2 is a digital image showing that collagen production by human fibroblasts is decreased by treatment with anti-CD70 (or CD27) and that this modulates the stimulatory effect of transforming growth factor (TGF)-β.

FIG. 3 is a digital image showing that CD70 antibody inhibits the production of other extracellular matrix (ECM) proteins by human fibroblasts.

FIG. 4 is a digital image of a Western blot documenting the presence of CD70 in human pulmonary fibroblasts.

FIG. 5 is a digital image of human abdominal skin treated with TGF-β (left panel) or TGF-β (right panel) and an anti-CD70 antibody.

FIG. 6 is a set of graphs showing that skin thickness in response to stimulation with TGF-β was decreased by sCD27.

FIG. 7 is a bar graph. Punch biopsies (3 mm diameter) were made of human foreskins that had been obtained during circumcisions and incubated with transforming growth factor (TGF)-β with and without anti-CD70 antibody.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Aug. 31, 2016, 3.67 KB], which is incorporated by reference herein.

### DETAILED DESCRIPTION

Disclosed herein are methods for the treatment of fibrosis in a subject using a CD70 agonist.

These methods can be used for the treatment of a variety of fibrotic conditions, such as, but not limited to, fibrotic conditions of the lung and the skin. The disclosed methods can be used to selectively target unwanted fibrosis in order to achieve a desired therapeutic outcome. The CD70 agonist can be administered systemically or locally. In some embodiments, the CD70 agonist is an antibody or antigen binding fragment thereof that specifically binds CD70 and stimulates a biological pathway mediated by CD70. In other embodiments, the agent is soluble CD27. In further embodiments, the CD70 agonist is a small molecule. In yet other embodiments, the subject is administered a nucleic acid molecule encoding the CD70 agonist, such as the sCD27, the antibody that specifically binds CD70, or the antigen binding fragment.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Agonist: A molecule that binds to a receptor and activates the receptor to produce a biological response. Whereas an agonist causes an action, an antagonist blocks the action of the agonist. Generally, an agonist is selective for a particular receptor, such that it activates the particular receptor to produce a biological response, but does not activate other receptors to product a biological response. The potency of an agonist is inversely related to its EC₅₀ value. The EC₅₀ can be measured for a given agonist by determining the concentration of agonist needed to elicit half of the maximum biological response of the agonist. An antibody is an agonist for a receptor, such as CD70, if binding of the antibody to CD70 produces a biological response that also results from the binding of a natural ligand, such as sCD27.

Animal: Living multi-cellular vertebrate organisms, a category that includes, for example, mammals and birds. The term mammal includes both human and non-human mammals. Similarly, the term “subject” includes both human and veterinary subjects.

Antibody: An immunoglobulin, antigen-binding fragment, or derivative thereof, that specifically binds and recognizes an analyte (antigen) such as CD70. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. An antibody is an agonist for CD70 if the antibody specifically binds to CD70 produces a biological response that results from the binding of the natural ligand, such as sCD27.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv is typically not decisive for scFvs. Thus, scFvs with both possible arrangements (V_{H}-domain-linker domain-V_{L}-domain; V_{L}-domain-linker domain-V_{H}-domain) may be used.

In a disulfide stabilized Fv (dsFv), the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are known, and an exemplary competition assay is provided herein.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6th ed., W. H. Freeman and Co., page 91 (2007)). In several embodiments, the V_{H} and V_{L} combine to specifically bind the antigen. In additional embodiments, only the V_{H} is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, MD, 1991; “Kabat” numbering scheme), Al-Lazikani et al. (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2n^{d} ed. Cold Spring Harbor Publications, New York (2013).

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1st Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008)

cDNA (complementary DNA): A piece of DNA lacking internal, non-coding segments (introns) and regulatory sequences that determine transcription. cDNA is synthesized in the laboratory by reverse transcription from messenger RNA extracted from cells.

CD27: A member of the tumor necrosis factor receptor superfamily that is important for generation and long-term maintenance of T cell immunity. CD27 binds to CD70, and plays a role in regulating B-cell activation and immunoglobulin synthesis. This receptor transduces signals that lead to the activation of NF-κB and MAPK8/JNK. Exemplary mRNA and protein sequences for murine CD27 are provided in GENBANK® Accession No. NM_001033126.2, Feb. 15, 2015 and GENBANK® Accession No. NP_001028298., Feb. 15, 2015. Exemplary mRNA and protein sequences for human CD27 are provided in GENBANK Accession Nos. NM_001242.4, Mar. 15, 2015 and GENBANK Accession No. NP_001233.1, Mar. 15, 2014. All of these sequences are incorporated by reference herein.

CD70: A cytokine that belongs to the tumor necrosis factor (TNF) ligand family and is a ligand for CD27. CD70 is a surface antigen on activated, but not on resting, T and B lymphocytes. CD70 induces proliferation of costimulated T cells, enhances the generation of cytolytic T cells, contributes to T cell activation, and plays a role in regulating B-cell activation, immunoglobulin synthesis, and cytotoxic function of natural killer cells. An exemplary mRNA and amino acid sequence of human CD70 is provided in GENBNAK® Accession No. NM_001252, Mar. 15, 2015 and GENBANK® Accession No. NP_001243.1, Mar. 15, 2015, both incorporated herein by reference. A “CD70 agonist” activates a biological pathway controlled by CD70 on a target cell, such as a fibroblast.

Collagen: Proteins that are found in the form of elongated fibrils in mammals that are mostly found in fibrous tissues such as tendon, ligament and skin, and is also abundant in cornea, cartilage, bone, blood vessels, the gut, and intervertebral disc. The tropocollagen or “collagen molecule” is a subunit of larger collagen aggregates such as fibrils. It is approximately 300 nm long and 1.5 nm in diameter, made up of three polypeptide strands (called alpha chains), each possessing the conformation of a left-handed helix. In type I collagen, each triple-helix associates into a right-handed super-super-coil that is referred to as the collagen microfibril.

Conservative variants: “Conservative” amino acid substitutions are those substitutions that do not substantially affect or decrease an activity or of a polypeptide, such as the ability of the polypeptide to bind CD70 and/or inhibit fibrosis. Specific, non-limiting examples of a conservative substitution include the following examples:

| Original Residue | Conservative Substitutions |
|---|---|
| Al | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that antibodies raised to the substituted polypeptide also immunoreact with the unsubstituted polypeptide. Non-conservative substitutions are those that reduce an activity, such as the ability of a protein to inhibit fibrosis.

Consists Essentially Of/Consists Of: With regard to a polypeptide, a polypeptide that consists essentially of a specified amino acid sequence if it does not include any additional amino acid residues. However, the polypeptide can include additional non-peptide components, such as labels (for example, fluorescent, radioactive, or solid particle labels), sugars or lipids. With regard to a polypeptide, a polypeptide that consists of a specified amino acid sequence does not include any additional amino acid residues, nor does it include additional biological components, such as nucleic acids lipids, sugars, nor does it include labels. A polypeptide that consists or consists essential of a specified amino acid sequence can be glycosylated or have an amide modification. With regard to a polynucleotide, a polynucleotide that consists essentially of a specified nucleic acid sequence if it does not include any additional nucleic acid residues. However, the polypeptide can include additional non-nucleic acid components, such as labels (for example, fluorescent, radioactive, or solid particle labels) or polypeptides. With regard to a polynucleotide, a polynucleotide that consists of a specified nucleic acid sequence does not include any additional nucleic acid residues, nor does it include additional biological components, such as proteins, nor does it include labels.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody, small molecule, or polypeptide in direct physical association with a cell.

Control: A reference standard. In some embodiments, the control is a sample obtained from a subject that is not treated or a sample obtained from a healthy subject. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Degenerate variant: A polynucleotide encoding a polypeptide, such as soluble CD27 or an antibody (or antigen binding fragment thereof) that specifically binds CD70, where the polynucleotide includes a sequence that is degenerate as a result of the genetic code. There are 20 natural amino acids, most of which are specified by more than one codon. Therefore, all degenerate nucleotide sequences are included in this disclosure as long as the amino acid sequence of the polypeptide encoded by the nucleotide sequence is unchanged.

Detecting: To identify the existence, presence, or fact of something. General methods of detecting are known to the skilled artisan and may be supplemented with the protocols and reagents disclosed herein. For example, fibrosis can be detected in a subject.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintainers of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like can be used. In one embodiment, when cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences.

Fibrosis: The formation or development of excess fibrous connective tissue in an organ or tissue as a reparative or reactive process, as opposed to a formation of fibrous tissue as a normal constituent of an organ or tissue. Skin and lungs are susceptible to fibrosis. Exemplary fibrotic conditions are scleroderma, idiopathic pulmonary fibrosis, morphea, fibrosis as a result of Graft-Versus-Host Disease (GVHD), keloid and hypertrophic scar, and subepithelial fibrosis, endomyocardial fibrosis, uterine fibrosis, myelofibrosis, retroperitoneal fibrosis, nephrogenic systemic fibrosis, scarring after surgery, asthma, cirrhosis/liver fibrosis, aberrant wound healing, glomerulonephritis, and multifocal fibrosclerosis.

Heterologous: Originating from separate genetic sources or species. Generally, an antibody that specifically binds to a protein of interest such as CD70 will not specifically bind to a heterologous protein. In specific, non-limiting examples, a polypeptide comprising sCD27 or an antibody that specifically binds CD70, and a heterologous amino acid sequence includes an Ig (such as sCD27-IgG₁), β-galactosidase, a maltose binding protein, and albumin, hepatitis B surface antigen, or a label.

Host cells: Cells in which a vector can be propagated and its DNA expressed. The cell may be prokaryotic or eukaryotic. The cell can be mammalian, such as a human cell. The term also includes any progeny of the subject host cell. It is understood that all progeny may not be identical to the parental cell since there may be mutations that occur during replication. However, such progeny are included when the term “host cell” is used.

Idiopathic Pulmonary Fibrosis: A condition also known as cryptogenic fibrosing alveolitis (CFA) that is a chronic, progressive form of lung disease characterized by fibrosis of the supporting framework (interstitium) of the lungs. By definition, the term is used only when the cause of the pulmonary fibrosis is unknown (“idiopathic”). When lung tissue from patients with IPF is examined under a microscope by a pathologist, it usually shows a characteristic set of histologic/pathologic features known as usual interstitial pneumonia (UIP). UIP is characterized by progressive scarring of both lung that involves the supporting framework (interstitium) of the lung.

Inhibiting or treating a disease: Inhibiting a disease, such as fibrosis, refers to inhibiting the full development of a disease. In several examples, inhibiting a disease refers to lessening symptoms of a fibrosis, such as the formation of scar tissue or an increase in range of motion or a decrease in pain. The inhibition of pulmonary fibrosis refers to lessening a symptom and/or improving lung function. “Treatment” refers to a therapeutic intervention that ameliorates a sign or symptom of a disease or pathological condition related to the disease, such as the fibrosis.

Isolated: An “isolated” biological component (such as a nucleic acid or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Keloid or keloidal scar: A type of scar, which, depending on its maturity, is composed of mainly either type III (early) or type I (late) collagen. It is a result of an overgrowth of granulation tissue (collagen type 3) at the site of a healed skin injury which is then slowly replaced by collagen type 1. Keloids are firm, rubbery lesions or shiny, fibrous nodules, and can vary from pink to flesh-colored or red to dark brown in color. A keloid scar is benign, non-contagious, and usually accompanied by severe itchiness, sharp pains, and changes in texture. In severe cases, it can affect movement of skin. Keloids are different than hypertrophic scars, which are raised scars that do not grow beyond the boundaries of the original wound.

Label: A detectable compound or composition that is conjugated directly or indirectly to another molecule to facilitate detection of that molecule. Specific, non-limiting examples of labels include fluorescent tags, enzymatic linkages, and radioactive isotopes.

Mammal: This term includes both human and non-human mammals. Similarly, the term “subject” includes both human and veterinary subjects.

Oligonucleotide: A linear polynucleotide sequence of up to about 100 nucleotide bases in length.

Open reading frame (ORF): A series of nucleotide triplets (codons) coding for amino acids without any internal termination codons. These sequences are usually translatable into a polypeptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence, such as a sequence that encodes a polypeptide, such as sCD27 or an antibody that specifically binds CD70. Generally, operably linked DNA sequences are contiguous and, where necessary to join two protein-coding regions, in the same reading frame.

Peptide Modifications: Polypeptides, such as sCD27 or an antibody (or antigen binding fragment thereof) that specifically binds CD70, include synthetic embodiments of polypeptides described herein. In addition, analogs (non-peptide organic molecules), derivatives (chemically functionalized polypeptide molecules obtained starting with the disclosed polypeptide sequences) and variants (homologs) of these proteins can be utilized in the methods described herein. Each polypeptide of this disclosure is comprised of a sequence of amino acids, which may be either L-and/or D-amino acids, naturally occurring and otherwise.

Peptides can be modified by a variety of chemical techniques to produce derivatives having essentially the same activity as the unmodified polypeptides, and optionally having other desirable properties. For example, carboxylic acid groups of the protein, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the polypeptide, whether amino-terminal or side chain, can be in the form of a pharmaceutically-acceptable acid addition salt, such as HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the polypeptide side chains may be converted to C₁-C₁₆ alkoxy or to a C₁-C₁₆ ester using well-recognized techniques. Phenyl and phenolic rings of the polypeptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the polypeptide side chains can be extended to homologous C₂-C₄ alkylenes. Thiols can be protected with any one of a number of well-recognized protecting groups, such as acetamide groups. Those skilled in the art will also recognize methods for introducing cyclic structures into the polypeptides of this invention to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the polypeptide backbone and component amino acid side chains, resulting in such peptido- and organomimetics of sCD27 or an antibody that specifically binds CD70 having measurable or enhanced ability to treat fibrosis. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition to achieve a desired effect in a subject being treated. For instance, this can be the amount necessary to induce an immune response, inhibit fibrosis, reduce scar volume or to measurably alter outward symptoms of the fibrotic condition. When administered to a subject, a dosage will generally be used that will achieve target tissue concentrations (for example, in skin cells or lung tissue) that has been shown to achieve an in vitro effect.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Peptide or Polypeptide: Any chain of amino acids, regardless of length or post-translational modification (e.g., glycosylation or phosphorylation). In one embodiment, the polypeptide is a sCD27 polypeptide. In another embodiment, a polypeptide is an antibody that specifically binds CD70. With regard to polypeptides, the word “about” indicates integer amounts, such as within one amino acid. Thus, in one example, a polypeptide “about” 50 amino acids in length is from 49 to 51 amino acids in length.

Post-translational modification: The modification of a newly formed protein; may involve deletion of amino acids, chemical modification of certain amino acids (for example, amidation, acetylation, phosphorylation, glycosylation, formation of pyroglutamate, oxidation/reduction of sulfa group on a methionine, or addition of similar small molecules) to certain amino acids

Probes and primers: A probe comprises an isolated nucleic acid attached to a detectable label or reporter molecule. Primers are short nucleic acids, preferably DNA oligonucleotides, of about 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example by polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers can be selected that comprise about 20, 25, 30, 35, 40, 50 or more consecutive nucleotides.

Purified: A sCD27 polypeptide and/or an antibody (or antigen binding fragment) that specifically binds CD70 disclosed herein can be purified (and/or synthesized) by any of the means known in the art (see, e.g., Guide to Protein Purification, ed. Deutscher, Meth. Enzymol. 185, Academic Press, San Diego, 1990; and Scopes, Protein Purification: Principles and Practice, Springer Verlag, N.Y., 1982). Substantial purification denotes purification from other proteins or cellular components. A substantially purified protein is at least about 60%, 70%, 80%, 90%, 95%, 98% or 99% pure. Thus, in one specific, non-limiting example, a substantially purified protein is 90% free of other proteins or cellular components.

Thus, the term purified does not require absolute purity; rather, it is intended as a relative term. For example, a purified nucleic acid is one in which the nucleic acid is more enriched than the nucleic acid in its natural environment within a cell. In additional embodiments, a nucleic acid or cell preparation is purified such that the nucleic acid or cell represents at least about 60% (such as, but not limited to, 70%, 80%, 90%, 95%, 98% or 99%) of the total nucleic acid or cell content of the preparation, respectively.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Scleroderma: A chronic autoimmune disease characterized by fibrosis (or hardening), vascular alterations, and autoantibodies. There are two major forms, one is a systemic form that includes limited cutaneous scleroderma mainly affects the hands, arms and face, although pulmonary hypertension is frequent. Diffuse cutaneous scleroderma (or systemic sclerosis) is rapidly progressing and affects a large area of the skin and one or more internal organs, frequently the kidneys, esophagus, heart and lungs. Systemic scleroderma in both of its forms can be fatal. The other form of scleroderma is a localized form that has two subtypes: morphea and linear scleroderma. The disclosed methods can be used to treat any form of scleroderma.

Selectively hybridize: Hybridization under moderately or highly stringent conditions that excludes non-related nucleotide sequences.

In nucleic acid hybridization reactions, the conditions used to achieve a particular level of stringency will vary, depending on the nature of the nucleic acids being hybridized. For example, the length, degree of complementarity, nucleotide sequence composition (for example, GC v. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a sCD27 polypeptide, or an antibody that specifically binds CD70, will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a sCD27 polypeptide, or an antibody (or antigen binding fragment) that specifically binds CD70, are typically characterized by possession of at least 75%, for example at least 80%, sequence identity counted over the full length alignment with the amino acid sequence of the a sCD27 polypeptide, or the antibody that specifically binds CD70, using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and can possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Therapeutically effective amount: A quantity of compound, such as a CD70 agonist, sufficient to achieve a desired effect in a subject being treated. For instance, this can be the amount necessary to treat or ameliorate fibrosis, such as skin or lung fibrosis, in a subject. In some embodiments, it is the amount necessary to treat a subject by a measurable amount over a period of time, or to measurably inhibit progression of disease, in a subject. In other embodiments, a therapeutically effective amount is the amount necessary to prophylactically inhibit a disease.

An effective amount of a CD70 agonist may be administered in a single dose, or in several doses, for example daily, during a course of treatment. However, the effective amount will be dependent on the compound applied, the subject being treated, the severity and type of the affliction, and the manner of administration of the compound.

Transduced: A transduced cell is a cell into which has been introduced a nucleic acid molecule by molecular biology techniques. As used herein, the term transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art. Vectors include plasmid vectors, including plasmids for expression in gram negative and gram positive bacterial cell. Exemplary vectors include those for expression in E. coli and Salmonella. Vectors also include viral vectors, such as, but are not limited to, retrovirus, orthopox, avipox, fowlpox, capripox, suipox, adenoviral, herpes virus, alpha virus, baculovirus, Sindbis virus, vaccinia virus and poliovirus vectors. Vectors also include vectors for expression in yeast cells and insect cells.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or peptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### CD70 Agonists

Disclosed herein are method for treating fibrosis using a CD70 agonist that binds and stimulates a CD70-controlled biological pathway. The cytokine receptor CD27 is a member of the tumor necrosis factor receptor (TFNR) superfamily, the members of which play a role in cell growth and differentiation, as well as apoptosis. The ligand for CD27 is CD70, which belongs to the tumor necrosis factor family of ligands. CD70 is a 193 amino acid polypeptide having a 20 amino acid hydrophilic N-terminal domain and a C-terminal domain containing 2 potential N-linked glycosylation sites (Goodwin, R.G. et al. (1993) Cell 73:447-56; Bowman et al. (1994) Immunol 152: 1756-61). Based on these features, CD70 was determined to be a type II transmembrane protein having an extracellular C-terminal portion.

In vivo, CD70 is transiently found on activated T and B lymphocytes and dendritic cells (Hintzen et al. (1994) J. Immunol. 152: 1762- 1773; Oshima et al. (1998) Int. Immunol. 10:517-26; Tesselaar et al. (2003) J. Immunol. 170:33-40). In addition to expression on normal cells, CD70 expression has been reported in different types of cancers including renal cell carcinomas, metastatic breast cancers, brain tumours, leukemias, lymphomas and nasopharyngeal carcinomas (Junker et al. (2005) Urol. 173:2150-3; Sloan et al. (2004) Am J Pathol. 164:315-23; Held-Feindt and Mentlein (2002) Int J Cancer 98:352- 6; Hishima et al. (2000) Am J Surg Pathol. 24:742-6; Lens et al. (1999) Br J Haematol. 106:491-503). It is disclosed herein that CD70 is expressed on fibroblasts.

The use of a CD70 agonist to treat fibrosis is disclosed herein. In some embodiments, a CD70 agonist is a soluble CD27 (sCD27) polypeptide or functional fragments thereof. An exemplary amino acid sequence of CD27 can be found on GENBANK® at Accession No. NP_001233, which is specifically incorporated by reference herein in its entirety as available Dec. 3, 2010. In one example, human CD27 has the amino acid sequence set forth below as SEQ ID NO: 1:

(SEQ ID NO: 1)
MARPHPWWLC VLGTLVGLSA TPAPKSCPER HYWAQGKLCC
QMCEPGTFLV KDCDQHRKAA QCDPCIPGVS FSPDHHTRPH
CESCRHCNSG LLVRNCTITA NAECACRNGW QCRDKECTEC
DPLPNPSLTA RSSQALSPHP QPTHLPYVSE MLEARTAGHM
QTLADFRQLP ARTLSTHWPP QRSLCSSDFI RILVIFSGMF
LVFTLAGALF LHQRRKYRSN KGESPVEPAE PCRYSCPREE
EGSTIPIQED YRKPEPACSP.

The expressed human CD27 (see, for example, NCBI Accession No. NP 001233, incorporated by reference herein) is a polypeptide of 260 amino acids in length that has a 20 amino acid secretion signal at the N-terminus. The CD27 antigen cytoplasmic tail, residues 213-260, binds to the N-terminus of the SIVA protein (also known as the apoptosis- inducing factor CD27BP; Siva-1, and Siva-2). The PIQEDYR motif (amino acids 246-252 of SEQ ID NO: 1) has been described as being important for NF-κB and SAPK/JNK activation and the interaction with TNF receptor-associated factor (TRAF) 2 and TRAFS.

In some embodiments, the sCD27 polypeptides contain an amino acid sequence that is at least 95% identical to the amino acid sequence set forth as SEQ ID NO: 1, such as at least 95%, at least 96%, at least 97%, at least 98%, at least 99%, or 100% identical to the amino acid sequence set forth as of SEQ ID NO: 1. In some embodiments, the sCD27 polypeptides contain an amino acid sequence that is at least 95% identical to the amino acid sequence set forth as residues 1-189 of SEQ ID NO: 1, such as at least 95%, at least 96%, at least 97%, at least 98%, at least 99%, or 100% identical to the amino acid sequence set forth as residues 1-189 of SEQ ID NO: 1. The sCD27 can include, or consist of the amino acid sequence set forth as SEQ ID NO: 1 or residues 1-189 of SEQ ID NO: 1. The sCD27 polypeptide binds to CD70. The polypeptide can include peptide modifications.

It is contemplated by this disclosure that additional soluble fragments of CD27 can be used in the disclosed methods; these fragments of CD27 are CD70 agonists. In one embodiment, the methods utilized amino acids 1 to 189 of the amino acid sequence set forth as SEQ ID NO: 1.

In some embodiments, the sCD27 includes amino acid residues 1 through 189 of the CD27 protein set forth as SEQ ID NO: 1. In some embodiments, a biologically active fragment of CD27 is at most 25, 50, 75, 100, 125, 150 or 175 consecutive amino acids of amino acids 1-189 of SEQ

ID NO: 1, wherein the functional fragments is soluble and is a CD70 agonist. In yet other embodiments, a functional fragment is amino acids 1 to 180, 181, 182, 183, 184, 185, 186, 187, 188, 189, 190, 191, 192, 193, 104, 105, 196, 197, 198, 199, 200, 201, 202, 203, 204, 205, 206, 207, 208, 209, 210, 211, 212, or 213 of SEQ ID NO: 1.

It is contemplated that sCD27 can contain one or more conservative amino acid substitutions, for example polymorphisms present in the population. “Conservative” amino acid substitutions are those substitutions that do not substantially affect or decrease an activity, such as binding to CD70. Thus the polypeptide can include at most about 1, at most about 2, at most about 5, and at most about 10, or at most about 15 conservative substitutions in the amino acid sequence provided above, see also GENBANK® Accession No. NP_001233 as available December 3, 2010, which is incorporated herein by reference in its entirety and given as SEQ ID NO: 1. Conservative variations also include the use of a substituted amino acid in place of an unsubstituted parent amino acid, provided that antibodies raised to the substituted polypeptide also immunoreact with the unsubstituted polypeptide. Non-conservative substitutions are those that reduce an activity, such as binding to CD70.

A sCD27 polypeptide also can be modified to increase the half-life of the antibody. For example, the polypeptide can be aminidated or linked to a heterologous proteins, such as an Fc domain. Other modifications include amino acid substitutions at non-binding sites, dimerization or trimerization, binding or fusion to a foreign particle (e.g., nanoparticle), and selective covalent modifications such as acetylation, nitrosylation, and citrulination.

An exemplary nucleic acid sequence encoding CD27 can be found on GENBANK® at Accession No. NM_001242.4, which is specifically incorporated by reference herein in its entirety as available December 3, 2010. In one example, a nucleic acid sequence encoding human CD27 has the nucleic acid sequence set forth below as SEQ ID NO: 2:

(SEQ ID NO: 2)
ATGGCACGGC CACATCCCTG GTGGCTGTGC GTTCTGGGGA
CCCTGGTGGG GCTCTCAGCT ACTCCAGCCC CCAAGAGCTG
CCCAGAGAGG CACTACTGGG CTCAGGGAAA GCTGTGCTGC
CAGATGTGTG AGCCAGGAAC ATTCCTCGTG AAGGACTGTG
ACCAGCATAG AAAGGCTGCT CAGTGTGATC CTTGCATACC
GGGGGTCTCC TTCTCTCCTG ACCACCACAC CCGGCCCCAC
TGTGAGAGCT GTCGGCACTG TAACTCTGGT CTTCTCGTTC
GCAACTGCAC CATCACTGCC AATGCTGAGT GTGCCTGTCG
CAATGGCTGG CAGTGCAGGG ACAAGGAGTG CACCGAGTGT
GATCCTCTTC CAAACCCTTC GCTGACCGCT CGGTCGTCTC
AGGCCCTGAG CCCACACCCT CAGCCCACCC ACTTACCTTA
TGTCAGTGAG ATGCTGGAGG CCAGGACAGC TGGGCACATG
CAGACTCTGG CTGACTTCAG GCAGCTGCCT GCCCGGACTC
TCTCTACCCA CTGGCCACCC CAAAGATCCC TGTGCAGCTC
CGATTTTATT CGCATCCTTG TGATCTTCTC TGGAATGTTC
CTTGTTTTCA CCCTGGCCGG GGCCCTGTTC CTCCATCAAC
GAAGGAAATA TAGATCAAAC AAAGGAGAAA GTCCTGTGGA
GCCTGCAGAG CCTTGTCGTT ACAGCTGCCC CAGGGAGGAG
GAGGGCAGCA CCATCCCCAT CCAGGAGGAT TACCGAAAAC
CGGAGCCTGC CTGCTCCCCC TGA.

Thus, one of skill in the art can readily produce nucleic acids encoding a sCD27. Polynucleotides include DNA, cDNA and RNA sequences which encode the peptide of interest. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., N.Y.).

CD70 agonists that are of use in the disclosed methods include antibodies that specifically bind CD70 and stimulate a biological pathway regulated by CD70, and antigen binding fragments of these antibodies. Antibodies that bind CD70 are known in the art and are commercially available, see Ancell, Catalog 222-020. Agonist antibodies of CD27 are also of use.

Exemplary antibodies and antigen binding fragments are disclosed, for example, in U.S. Published Patent Application No. 2010/0150950, incorporated herein by reference. Thus publication discloses human antibodies that bind CD70 with a high affinity and are function stimulating. U.S. Pat. No. 8,535,678 also discloses antibodies that that specifically bind CD70.

Additional antibodies are commercially available, such as ARGX-110 (arGEN-X) and SGN-CD70A (Seattle Genetics). Other monoclonal antibodies are commercially available, such as MAPF112, clone TAN 1-7 form Millipore. Another antibody of use is an agonist antibody, such as CDX-1127 (see Ancell et al., J. Clin. Oncol. 32:5s, 2014).

In several embodiments, the antibody or antigen binding fragment can specifically bind CD70 with an affinity (e.g., measured by K_{D}) of no more than 1.0×10⁻⁸M, no more than 5.0×10⁻⁸M, no more than 1.0×10⁻⁹M, no more than 5.0×10⁻⁹M, no more than 1.0×10⁻¹⁰ M, no more than 5.0×10⁻¹⁰M, or no more than 1.0×10⁻¹¹M. K_{D} can be measured, for example, by a radiolabeled antigen binding assay (RIA) performed with the Fab version of an antibody of interest and its antigen using known methods. In another assay, K_{D} can be measured using surface plasmon resonance assays using a BIACORE®-2000 or a BIACORE®-3000 (BlAcore, Inc., Piscataway, N.J.) at 25° C. with immobilized antigen CMS chips at ˜10 response units (RU).

The antibodies can be from any species. The antibodies can be fully human antibodies or chimeric antibodies. The antibody or antigen binding fragment can be a human antibody or fragment thereof. The antibody or antigen binding fragment can be a murine antibody. Chimeric antibodies are also of use in the disclosed methods. In some embodiments, the antibody or antigen binding fragment includes any suitable framework region, such as (but not limited to) a human framework region. Human framework regions, and mutations that can be made in a human antibody framework regions, are known in the art (see, for example, in U.S. Pat. No. 5,585,089, which is incorporated herein by reference). Alternatively, a heterologous framework region, such as, but not limited to a mouse or monkey framework region, can be included in the antibody (See, for example, Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech.12:437, 1992; and Singer et al., J. Immunol.150:2844, 1993).

An antibody that specifically binds CD70 can be of any isotype. The antibody can be, for example, an IgM or an IgG antibody, such as IgG₁, IgG₂, IgG₃, or IgG₄. The class of an antibody that specifically binds CD70 can be switched with another. In one aspect, a nucleic acid molecule encoding V_{L} or V_{H} is isolated using methods well-known in the art, such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds CD70, that was originally IgM may be class switched to an IgG. Class switching can be used to convert one IgG subclass to another, such as from IgG₁ to IgG₂, IgG₃, or IgG₄.

Antigen binding fragments are encompassed by the present disclosure, such as Fab, F(ab′)₂, and Fv which include a heavy chain and V_{L} and specifically bind CD70. These antibody fragments retain the ability to selectively bind with the antigen and are “antigen-binding” fragments. Non-limiting examples of such fragments include:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the V_{L} and V_{L} expressed as two chains; and

(5) Single chain antibody (such as scFv), defined as a genetically engineered molecule containing the VH and the V_{L} linked by a suitable polypeptide linker as a genetically fused single chain molecule (see, e.g., Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.

(6) A dimer of a single chain antibody (scFV₂), defined as a dimer of a scFV. This has also been termed a “miniantibody.”

Methods of making these fragments are known in the art (see for example, Harlow and Lane, Antibodies: A Laboratory Manual, 2^{nd}, Cold Spring Harbor Laboratory, N.Y., 2013).

In some embodiments, the antigen binding fragment can be an Fv antibody, which is typically about 25 kDa and contain a complete antigen-binding site with three CDRs per each heavy chain and each light chain. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the V_{H} and the V_{L} are chemically linked by disulfide bonds.

In an additional example, the Fv fragments include V_{H} and V_{L} chains connected by a peptide linker. These single-chain antigen binding proteins (scFv) can be prepared by constructing a nucleic acid molecule encoding the V_{H} and V_{L} domains connected by an oligonucleotide. The nucleic acid molecule is inserted into an expression vector, which is subsequently introduced into a host cell such as a mammalian cell. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; Ahmad et al., Clin. Dev. Immunol., 2012, doi:10.1155/2012/980250; Marbry, IDrugs, 13:543-549, 2010). Dimers of a single chain antibody (scFV₂), are also contemplated.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Antigen binding single V_{H} domains, called domain antibodies (dAb), have also been identified from a library of murine V_{H} genes amplified from genomic DNA of immunized mice (Ward et al. Nature 341:544-546, 1989). Human single immunoglobulin variable domain polypeptides capable of binding antigen with high affinity have also been described (see, for example, PCT Publication Nos. WO 2005/035572 and WO 2003/002609). The CDRs disclosed herein can also be included in a dAb.

In certain embodiments, amino acid sequence variants of the antibodies provided herein are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and the framework regions Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules Amino acid substitutions (such as conservative amino acid substitutions) can be made in the V_{H} and the V_{L} regions, for example to increase production yield or solubility.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In certain embodiments of the variant V_{H} and V_{L} sequences provided above, each CDR either is unaltered, or contains no more than one, two or three amino acid substitutions.

To increase binding affinity of the antibody for CD70, the V_{L} and V_{H} segments can be randomly mutated, such as within HCDR3 region or the LCDR3 region, in a process analogous to the in vivo somatic mutation process responsible for affinity maturation of antibodies during a natural immune response. Thus, in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for CD70. Methods of in vitro affinity maturation are known (see, e.g., Chowdhury, Methods Mol. Biol. 207:179-196 (2008), and Hoogenboom et al. in Methods in Molecular Biology 178:1-37 (O'Brien et al., ed., Human Press, Totowa, N.J., (2001)).

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

In certain embodiments, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. TIBTECH 15:26-32 (1997). The oligosaccharide may include various carbohydrates, e g , mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec 13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In several embodiments, the constant region of the antibody includes one or more amino acid substitutions to optimize in vivo half-life of the antibody. The serum half-life of IgG Abs is regulated by the neonatal Fc receptor (FcRn). Thus, in several embodiments, the antibody includes an amino acid substitution that increases binding to the FcRn. Several such substitutions are known to the person of ordinary skill in the art, such as substitutions at IgG constant regions T250Q and M428L (see, e.g., Hinton et al., J. Immunol., 176:346-356, 2006); M428L and N434S (the “LS” mutation, see, e.g., Zalevsky, et al., Nature Biotechnology, 28:157-159, 2010); N434A (see, e.g., Petkova et al., Int. Immunol., 18:1759-1769, 2006); T307A, E380A, and N434A (see, e.g., Petkova et al., Int. Immunol., 18:1759-1769, 2006); and M252Y, S254T, and T256E (see, e.g., Dall'Acqua et al., J. Biol. Chem., 281:23514-23524, 2006). The disclosed antibodies and antigen binding fragments can be linked to a Fc polypeptide including any of the substitutions listed above, for example, the Fc polypeptide can include the M428L and N434S substitutions.

CD70 agonists also include molecules that are identified from large libraries of both natural product or synthetic (or semi-synthetic) extracts or chemical libraries according to methods known in the art. The screening methods that detect skin thickness or any biological pathway regulated by CD70 are useful for identifying compounds from a variety of sources for activity. The initial screens may be performed using a diverse library of compounds, a variety of other compounds and compound libraries. Thus, molecules that bind CD70, molecules that induce the expression of CD70, and molecules that induce the activity of CD70 can be utilized. These small molecules can be identified from combinatorial libraries, natural product libraries, or other small molecule libraries. In addition, a CD70 agonist can be identified as compounds from commercial sources, as well as commercially available analogs of identified inhibitors.

The precise source of test extracts or compounds is not critical to the identification of CD70 agonists. Accordingly, CD70 agonists can be identified from virtually any number of chemical extracts or compounds. Examples of such extracts or compounds that can be CD70 agonists include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). PD-1 antagonists can be identified from synthetic compound libraries that are commercially available from a number of companies including Maybridge Chemical Co. (Trevillet, Cornwall, UK), Comgenex (Princeton, N. J.), Brandon Associates (Merrimack, N.H.), and Microsource (New Milford, Conn.). PD-1 antagonists can be identified from a rare chemical library, such as the library that is available from Aldrich (Milwaukee, Wis.). CD70 agonists can be identified in libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Natural and synthetically produced libraries and compounds are readily modified through conventional chemical, physical, and biochemical means.

Useful compounds may be found within numerous chemical classes, though typically they are organic compounds, including small organic compounds. Small organic compounds have a molecular weight of more than 50 yet less than about 2,500 daltons, such as less than about 750 or less than about 350 daltons and can be utilized in the methods disclosed herein. Exemplary classes include heterocycles, peptides, saccharides, steroids, and the like. The compounds may be modified to enhance efficacy, stability, pharmaceutical compatibility, and the like. In several embodiments, compounds of use has a Kd for CD70 of less than 1 nM, less than 10 nm, less than 1 μM, less than 10 μM, or less than 1 mM.

### Polynucleotides Encoding a sCD27 Polypeptide, or an Antibody that Specifically Binds CD70 and Host Cells

Polynucleotides encoding a CD70 agonist, such as a sCD27 polypeptide, an antibody that specifically binds CD70, or an antigen binding fragment of the antibody, are also of use in the disclosed methods. These polynucleotides include DNA, cDNA and RNA sequences which encode the peptide of interest. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or

CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

A nucleic acid encoding a CD70 agonist, such as sCD27 polypeptide, an antibody that specifically binds CD70, or an antigen binding fragment of the antibody, can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding a CD70 agonist, such as a sCD27 polypeptide, an antibody that specifically binds CD70, or an antigen binding fragment of the antibody, include a recombinant DNA which is incorporated into a vector in an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

In one embodiment, vectors are used for expression in yeast such as S. cerevisiae or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2 μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The polynucleotides can also be designed to express in insect cells.

The CD70 agonist, such as the sCD27 polypeptide, the antibody that specifically binds CD70, or the antigen binding fragment of the antibody, can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared encoding the CD70 agonist, such as the sCD27 polypeptide, the antibody that specifically binds CD70, or the antigen binding fragment of the antibody. A number of viral vectors have been constructed, including polyoma, SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739).

Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Thus, in one embodiment, the polynucleotide encoding the CD70 agonist, such as the sCD27 polypeptide, the antibody that specifically binds CD70, or the antigen binding fragment of the antibody, is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like.

Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Examples of pox viral vectors for expression as described for example, in U.S. Pat. No. 6,165,460, which is incorporated herein by reference. Other viral vectors that can be used include other DNA viruses such as herpes virus and adenoviruses, and RNA viruses such as retroviruses and polio.

Suitable vectors are disclosed, for example, in U.S. Pat. No. 6,998,252, which is incorporated herein by reference. In one example, a recombinant poxvirus, such as a recombinant vaccinia virus is synthetically modified by insertion of a chimeric gene containing vaccinia regulatory sequences or DNA sequences functionally equivalent thereto flanking DNA sequences which in nature are not contiguous with the flanking vaccinia regulatory DNA sequences that encode the CD70 agonist, such as the sCD27 polypeptide, the antibody that specifically binds CD70, or the antigen binding fragment of the antibody. The recombinant virus containing such a chimeric gene is effective at expressing the CD70 agonist. In one example, the vaccine viral vector comprises (A) a segment comprised of (i) a first DNA sequence encoding the CD70 agonist, such as the sCD27 polypeptide, the antibody that specifically binds CD70, or the antigen binding fragment of the antibody and (ii) a poxvirus promoter, wherein the poxvirus promoter is adjacent to and exerts transcriptional control over the DNA sequence encoding the CD70 agonist; and, flanking said segment, (B) DNA from a nonessential region of a poxvirus genome. The viral vector can encode a selectable marker. In one example, the poxvirus includes, for example, a thymidine kinase gene (see U.S. Pat. No. 6,998,252, which is incorporated herein by reference).

Poxviral vectors that encode the CD70 agonist, such as the sCD27 polypeptide, the antibody that specifically binds CD70, or the antigen binding fragment of the antibody include at least one expression control element operationally linked to the nucleic acid sequence encoding the CD70 agonist. The expression control elements are inserted in the poxviral vector to control and regulate the expression of the nucleic acid sequence. Examples of expression control elements of use in these vectors include, but are not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the CD70 agonist, such as the sCD27 polypeptide, the antibody that specifically binds CD70, or the antigen binding fragment of the antibody, in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the CD70 agonist, such as the sCD27 polypeptide, the antibody that specifically binds CD70, or the antigen binding fragment of the antibody, are known in the art. Such techniques involve, for example, homologous recombination between the viral DNA sequences flanking the DNA sequence in a donor plasmid and homologous sequences present in the parental virus (Mackett et al., 1982, Proc. Natl. Acad. Sci. USA 79:7415-7419). In particular, recombinant viral vectors such as a poxvirus vector can be used in delivering the gene. The vector can be constructed for example by steps known in the art, such as steps analogous to the methods for creating synthetic recombinants of the fowlpox virus described in U.S. Pat. No. 5,093,258, incorporated herein by reference. Other techniques include using a unique restriction endonuclease site that is naturally present or artificially inserted in the parental viral vector to insert the heterologous DNA.

DNA sequences encoding the CD70 agonist, such as the sCD27 polypeptide, the antibody that specifically binds CD70, or the antigen binding fragment of the antibody, can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

As noted above, a polynucleotide sequence encoding the CD70 agonist, such as the sCD27 polypeptide, the antibody that specifically binds CD70, or the antigen binding fragment of the antibody, can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signals for introns, maintainers of the correct reading frame of a gene to permit proper translation of mRNA, and stop codons.

Hosts cells can include microbial, yeast, insect and mammalian host cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. As discussed above, techniques for the transformation of yeast cells, such as polyethylene glycol transformation, protoplast transformation and gene guns are also known in the art (see Gietz and Woods Methods in Enzymology 350: 87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding the CD70 agonist, such as the sCD27 polypeptide, the antibody that specifically binds CD70, or the antigen binding fragment of the antibody, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

### Therapeutic Methods and Pharmaceutical Compositions

As disclosed herein, a CD70 agonist, such as a sCD27 polypeptide, an antibody that specifically binds CD70, an antigen binding fragment of the antibody, or a small molecule can be used to treat fibrosis and/or reduce skin thickness. In several examples, the CD70 agonist, such as the sCD27 polypeptide, the antibody that specifically binds CD70, the antigen binding fragment of the antibody, small molecule or nucleic acid encoding a CD70 agonist, are of use to decrease fibrosis in a subject. Thus, in several embodiments, the methods include administering to a subject a therapeutically effective amount of one or more of a sCD27 polypeptide, an antibody that specifically binds CD70, an antigen binding fragment thereof, a small molecule, and/or polynucleotide encoding a CD70 agonist, in order to decrease fibrosis. In some embodiments, the CD70 agonist can be administered as a unit dose. The CD70 agonist can be administered systemically or locally. The subject can be a human or a veterinary subject.

Suitable subjects include those with a fibrosis of the skin or lungs, but fibrosis of any tissue can be treated using the methods disclosed herein. In one example, the subject has scleroderma. In other examples, the subject has idiopathic pulmonary fibrosis, morphea, fibrosis as a result of Graft-Versus-Host Disease (GVHD), a keloid or hypertrophic scar, subepithelial fibrosis, endomyocardial fibrosis, uterine fibrosis, myelofibrosis, retroperitoneal fibrosis, nephrogenic systemic fibrosis, scarring after surgery, asthma, cirrhosis/liver fibrosis, aberrant wound healing, glomerulonephritis, and multifocal fibrosclerosis.

In some embodiments, the subject has scleroderma. Thus, the methods are of use to treat the systemic form of scleroderma, such as limited cutaneous scleroderma-or diffuse cutaneous scleroderma (or systemic sclerosis). The methods can be used to treat the localized form of scleroderma, including morphea and linear scleroderma.

In other embodiments, the subject has pulmonary fibrosis. The subject can have pulmonary fibrosis secondary to another disease condition. The subject can have an interstitial lung disease. Pulmonary fibrosis can be caused by many conditions including chronic inflammatory processes (sarcoidosis, Wegener's granulomatosis), infections, environmental agents (asbestos, silica, exposure to certain gases), exposure to ionizing radiation (such as radiation therapy to treat tumors of the chest), chronic conditions (lupus, rheumatoid arthritis), and medications (such as from treatment with amiodarone, bleomycin (pingyangmycin), busulfan, methotrexate, apomorphine and nitrofurantoin). Fibrosis in any of these subjects can be treated using the disclosed methods. In some embodiments, the subject has idiopathic pulmonary fibrosis.

The methods can include selecting a subject in need of treatment, such as a subject any subject with fibrosis. The fibrosis can be of any organ. The subject can have a fibrotic disease, such as scleroderma, pulmonary fibrosis, idiopathic pulmonary fibrosis, morphea, a keloid scar, a hypertrophic scar, or subepithelial fibrosis. In exemplary applications, compositions are administered to a subject having a fibrotic disease, such as scleroderma, pulmonary fibrosis idiopathic pulmonary fibrosis, morphea, a keloid scar, a hypertrophic scar, or subepithelial fibrosis, or any of the disorders listed above, in an amount sufficient to reduce the fibrosis. The fibrosis can be the result of the end stage of many inflammatory disease processes, including autoimmune diseases. Fibrosis also includes fibrosis mediastinits, constrictive pericarditis, myocardial fibrosis, renal, liver, and bone marrow fibrosis.

Amounts effective for this use will depend upon the severity of the disease, the general state of the patient's health, and the robustness of the patient's immune system. In one example, a therapeutically effective amount of the compound is that which provides either subjective relief of a symptom(s) or an objectively identifiable improvement as noted by the clinician or other qualified observer.

In further embodiments, treatment with a CD70 agonist, such as a sCD27 polypeptide, an antibody that specifically binds CD70, an antigen binding fragment of the antibody, or a small molecule is administered in multiple doses over time, such as for at least 3 weeks, 6 weeks, 9 weeks, 12 weeks, 15 weeks, 18 weeks, 21 weeks, 24 weeks, 27 weeks, 30 weeks, 33 weeks, 36 weeks or 48 weeks. In other embodiments, treatment is for 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less.

In some embodiments, a method is provided herein for decreasing skin thickness. The method includes administering a therapeutically effective amount of a CD70 agonist, such as a sCD27 polypeptide, an antibody that specifically binds CD70, an antigen binding fragment of the antibody, and/or a small molecule, thereby decreasing skin thickness. The disclosed methods can reduce skin thickness by about 5%, 10%, 15%, 20%, 2.5%, 30%, 40%, 50%, 60%, 70%, 80%, 90%, 95%, 96%, 97%, 98%, 99% or 100% as compared to a control, such as an untreated subject.

In another embodiment, a method is provided for decreasing lung fibrosis. The method includes administering a therapeutically effective amount of a CD70 agonist, such as a sCD27 polypeptide, an antibody that specifically binds CD70, an antigen binding fragment of the antibody, and/or a small molecule, thereby decreasing lung fibrosis. In some embodiments, the subject has idiopathic lung fibrosis.

Numerous pulmonary function parameters known in the art can be used to determine an effective amount of a CD70 agonist, such as a sCD27 polypeptide, an antibody that specifically binds CD70, an antigen binding fragment of the antibody, and/or a small molecule, to reduce, stabilize or reverse a pathologic rate of decline in one or more pulmonary function parameters. These parameters can also be used to monitor patient response to the CD70 agonist, such as the sCD27 polypeptide, the antibody that specifically binds CD70, and/or the antigen binding fragment of the antibody. These pulmonary function parameters include the following:

1. Vital capacity (VC): The total volume of air that can be moved in and out of the lungs. VC is equal to the combined inspiratory reserve volume, tidal volume, and expiratory reserve volume.

2. Forced vital capacity (FVC): The vital capacity from a maximally forced expiratory effort.

3. FVC % predicted: A subject's measured FVC expressed as the percentage of the predicted FVC for the subject. Generally, all FVC % predicted values are absolute values and not relative values.

4. Residual volume (RV): The volume of air remaining in the lungs after a maximal exhalation.

5. Forced expiratory volume (FEV): The expiratory volume of air from a maximally forced expiratory effort, usually measured over a set period of time, such as 1 second or 6 seconds.

6. Forced inspiratory flow (FIF): The inspiratory volume of air from a maximally forced inspiratory effort, usually measured over a set period of time, such as 1 second or 6 seconds.

7. Peak expiratory flow rate (PEFR): The highest forced expiratory flow rate.

8. Inspiratory reserve volume (IRV): The maximal volume that can be inhaled after a normal inspiration, measured from the end-inspiratory level.

9. Tidal volume (TV): The volume of air inhaled or exhaled during one respiratory cycle, typically measured at rest.

10. Inspiratory capacity (IC): The sum of the inspiratory reserve volume and the tidal volume.

11. Functional residual capacity (FRC): The sum of the expiratory reserve volume and the residual volume. Typically, FRC represents the volume of air in the lungs at the end of a normal expiration.

12. Total lung capacity (TLC): The sum of the vital capacity arid residual volume that represents the total volume of air that can be contained in the lung.

13 Expiratory reserve volume (ERV): The maximal volume of air that can be exhaled after a normal expiration, measured from the end-expiratory position. 14. Maximum volume ventilation (MVV): The volume of air expired in a specified time period during repetitive maximal effort.

15. FEV: FVG ratio: The ratio between forced expiratory volume in one second and forced vital capacity.

Many of these pulmonary function parameters are readily obtainable through the use of a spirometer as is well-known in the art. Residual volume can be obtained through indirect methods such as radiographic planimetry, body plethysmography, closed circuit dilution (including the helium dilution technique), and nitrogen washout. Any of these parameters can be used to document the effectiveness of the disclosed methods.

Lung capacity and associated pulmonary function parameters naturally decline due to aging. Numerous normal populations have been studied and the rate of decline of lung capacity and various pulmonary function parameters have been calculated and are readily available in the art. (Crapo et al. Am. Rev. Respir. Bis. 123:659-664.) For example, a 65-year-old Caucasian male who is 183 cm tall has a predicted FVC of 4.95 liters. At age 66, this same male has a predicted FVC of 4.92 liters. This difference of 0.03 liters represents the expected decline due to aging by 1 year. Similarly, a 62-year-old Caucasian woman who is 167 cm has a predicted FVC of 2.67 liters. At age 63, this same female has a predicted FVC of 2.64 liters. This difference of 0.03 liters represents the expected decline due to aging by 1 year.

In contrast to the natural decline due to aging, subjects with idiopathic pulmonary fibrosis have an abnormally steep rate of decline in lung capacity or in one or more pulmonary function parameters, and thus have a “pathologic rate of decline.” A “pathologic rate of decline” is a rate of decline in lung capacity or in one or more pulmonary function parameters that is at least about 5% greater than the decline due to normal aging. In some embodiments, a pathologic rate of decline is at least about 5%, 10%, 15%, 20%, 2.5%, 30%, 40%, 50%, 60%, 70%, 80%, 90%, 100%, 125%, 150%, 200%, .300%, 400%, 500%ₛ 600%, 700%, 800% or 1000% greater than the predicted rate of decline for a normal person of similar matched race or ethnicity, gender, age, height, and weight. Rates of decline can be expressed as the change from baseline per 1 week, 2 weeks, 4 weeks, 8 weeks, 12 weeks, 2 weeks, 36 weeks, 48 weeks, or 12 months. In particular embodiments, the pathologic rate of decline in lung capacity is the change in forced vital capacity (FVC) from baseline of at least about −0.05 liters, −0.10 liters, −0.15 liters, −0.20 liters or −0.25 liters per 12 months. In other embodiments, the pathological rate of decline is the change from baseline forced vital capacity percent (FVC %) predicted of at least about −2%, −3%, −4%, −5%,. −6%, −7%, −8% or −10% per 12 months. These subjects can be selected for treatment using the disclosed methods.

In some embodiments, a method is provided for increasing FVC % predicted in a subject with IPF by administering an effective amount of a CD70 agonist, such as a sCD27 polypeptide, an antibody that specifically binds CD70, an antigen binding fragment of the antibody, and/or a small molecule. In further embodiments, treatment with an effective amount of a CD70 agonist, such as a sCD27 polypeptide, an antibody that specifically binds CD70, an antigen binding fragment of the antibody, and/or a small molecule increases FVC % predicted by at least 0.5%, 1%, 1.5%, 2.0%, 2.5%, 3.0%, 4.0%, 5.0%, 6.0%, 7.0%, 8.0%, 9.0%, 10%, 15%, 20%, 30%, 40% or 50% compared to base-line.

In some embodiments, if a subject with IPF has a baseline FVC % predicted of 65%, treatment with a CD70 agonist, such as a sCD27 polypeptide, an antibody that specifically binds CD70, and/or an antigen binding fragment of the antibody, it can increase the subject's FVC % predicted to 66.5% at week 36 post-initiation of therapy. In some embodiments, a method is provided for reducing, stabilizing, or reversing a pathologic rate of decline in one or more pulmonary function parameters, comprising the administration of an effective amount of a CD70 agonist, such as a sCD27 polypeptide, an antibody that specifically binds CD70, an antigen binding fragment of the antibody and/or a small molecule. In further embodiments, treatment with an effective amount of a CD70 agonist, such as a sCD27 polypeptide, an antibody that specifically binds CD70, an antigen binding fragment of the antibody, and/or a small molecule reduces the pathologic rate of decline of one or more pulmonary function parameters by at least 5%, 10%, 15%, 20%, 30%, 40%, 50%, 60%, 80%, or 100%. In particular embodiments, the pulmonary function parameter is FVC % predicted. In further embodiments, the reduction, stabilization or reversal in the pathologic rate of decline is achieved in 3 weeks or less, 6 weeks or less, 9 weeks or less, 12 weeks or less, 18 weeks or less, 24 weeks or less, 36 weeks or less, 48 weeks or less, 12 months or less, 16 months or less, 20 months or less, or 24 months or less from starting treatment.

A CD70 agonist, such as a sCD27 polypeptide, an antibody that specifically binds CD70, an antigen binding fragment of the antibody, a small molecule, or a nucleic acid encoding a CD70 agonist can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, PA, 1995) either locally or systemically, such as by intradermal, intrathecal, intramuscular, subcutaneous, intraperitoneal or intravenous injection, but even oral, nasal, transdermal or anal administration is contemplated. In one embodiment, administration is by subcutaneous, intradermal, or intramuscular injection. In another embodiment, administration is by intraperitoneal or intrathecal administration. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle. (see, e.g., Banga, supra).

For treatment of the skin, a therapeutically effective amount of a CD70 agonist, such as sCD27 polypeptide, an antibody that specifically binds CD70, an antigen binding fragment of the antibody, a small molecule, or a nucleic acid encoding a CD70 agonist, can be locally administered to the affected area of the skin, such as in the form of an ointment. In one embodiment, the ointment is an entirely homogenous semi-solid external agent with a firmness appropriate for easy application to the skin. Such an ointment can include fats, fatty oils, lanoline, Vaseline, paraffin, wax, hard ointments, resins, plastics, glycols, higher alcohols, glycerol, water or emulsifier and a suspending agent. Using these ingredients as a base, a decoy compound can be evenly mixed. Depending on the base, the mixture can be in the form of an oleaginous ointment, an emulsified ointment, or a water-soluble ointment oleaginous ointments use bases such as plant and animal oils and fats, wax, Vaseline and liquid paraffin. Emulsified ointments are comprised of an oleaginous substance and water, emulsified with an emulsifier. They can take either an oil-in-water form (O/W) or a water-in-oil-form (W/O). The oil-in-water form (O/W) can be a hydrophilic ointment. The water-in-oil form (W/O) initially lacks an aqueous phase and can include hydrophilic Vaseline and purified lanoline, or it can contain a water-absorption ointment (including an aqueous phase) and hydrated lanoline. A water-soluble ointment can contain a completely water-soluble Macrogol base as its main ingredient.

Pharmaceutically acceptable carriers include a petroleum jelly, such as VASELINE®, wherein the petroleum jelly contains 5% stearyl alcohol, or petroleum jelly alone, or petroleum jelly containing liquid paraffin. Such carriers enable pharmaceutical compositions to be prescribed in forms appropriate for consumption, such as tablets, pills, sugar-coated agents, capsules, liquid preparations, gels, ointments, syrups, slurries, and suspensions. When locally administered into cells in an affected area or a tissue of interest, the CD70 agonist, or polynucleotide encoding a CD70 agonist, can be administered in a composition that contains a synthetic or natural hydrophilic polymer as the carrier. Examples of such polymers include hydroxypropyl cellulose and polyethylene glycol. One or more CD70 agonists, or polynucleotide encoding the CD70 agonist, can be mixed with a hydrophilic polymer in an appropriate solvent. The solvent is then removed by methods such as air-drying, and the remainder is then shaped into a desired form (for example, a sheet) and applied to the target site. Formulations containing such hydrophilic polymers keep well as they have a low water-content. At the time of use, they absorb water, becoming gels that also store well. In the case of sheets, the firmness can be adjusted by mixing a polyhydric alcohol with a hydrophilic polymer similar to those above, such as cellulose, starch and its derivatives, or synthetic polymeric compounds. Hydrophilic sheets thus formed can be used. A therapeutically effective amount of one or more CD70 agonists, or polynucleotide encoding the agonist can also be incorporated into bandages and dressings.

For administration by inhalation, the CD70 agonist, such as the sCD27 polypeptide, an antibody that specifically binds CD70, an antigen binding fragment of the antibody, a small molecule, or polynucleotide encoding a CD70 agonist, can be conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges for use in an inhaler or insufflator can be formulated containing a powder mix of the compound and a suitable powder base such as lactose or starch.

In some embodiments, a CD70 agonist, such as a sCD27 polypeptide, an antibody that specifically binds CD70, an antigen binding fragment of the antibody, a small molecule, or a nucleic acid encoding a CD70 agonist can be administered by inhalation. For example, the CD70 agonist can be administered in an aerosolized form, such as using a nebulizer or a metered dose inhaler. In some embodiments, technologies of use include micropump nebulizers (such as the AEROGEN GO® system), jet nebulizers designed to produce large fine particle fractions (such as the PARI LC STAR®), jet nebulizers developing less shear during atomization (such as the HUDSON MICROMIST®), and ultrasonic nebulizers (such as the DeVilbiss ULTRA-NEB®).

The CD70 agonist can be dissolved in a carrier, such as saline, and atomized using the devices above. The associated aerosols can be collected, for example, using a NEXT GENERATION IMPACTOR® (NGI) (MSP Corp., Shoreview, Minn.), which uses a series of aerodynamic stages to separate and collect the aerosol into separate fractions based on droplet size. Since droplet size is the primary determinant of deposition location in the lungs, this device allows us to specifically isolate the portion of the liquid aerosol that will deposit in the small airways and alveoli.

Aerosol particle size is often expressed in terms of mass median aerodynamic diameter (MMAD), a parameter that is based on particle size, shape, and density. For a spherical particle, MMAD is equal to MMD (p^{1/2}), in which MMD is mass median diameter and r is the bulk density. For a non-spherical particle, MMAD is equal to MMD (p/x)^{1/2}, in which X is the shape factor. Thus, particles with larger than unit density will have actual diameters smaller than their MMAD.

The site of particle deposition within the respiratory tract is demarcated based on particle size. In one example, particles of about 1 to about 500 microns are utilized, such as particles of about 25 to about 250 microns, or about 10 to about 25 microns are utilized. In other embodiments, particles of about 1 to 50 microns are utilized. For use in a metered dose inhaler, for administration to lungs particles of less than about 10 microns, such as particles of about 2 to about 8 microns, such as about 1 to about 5 microns, such as particles of 2 to 3 microns, can be utilized.

A therapeutically effective amount of a CD70 agonist, such as a sCD27 polypeptide, an antibody that specifically binds CD70, an antigen binding fragment of the antibody, a small molecule, or polynucleotide a CD70 agonist, can be administered in the pharmaceutically acceptable carrier. Pharmacologically acceptable carriers (e.g., physiologically or pharmaceutically acceptable carriers) are well known in the art, and include, but are not limited to buffered solutions as a physiological pH (e.g. from a pH of about 7.0 to about 8.0, or at a pH of about 7.4). One specific, non-limiting example of a physiologically compatible buffered solution is phosphate buffered saline. Other pharmacologically acceptable carriers include penetrants, which are particularly suitable for pharmaceutical formulations that are intended to be topically applied (for example in the application of surgical wounds to promote healing).

The pharmacological compositions disclosed herein facilitate the use of a CD70 agonist, such as sCD27 polypeptide, an antibody that specifically binds CD70, an antigen binding fragment of the antibody, a small molecule, or a nucleic acid encoding a CD70 agonist, either in vivo or ex vivo, to decrease fibrosis. Such a composition can be suitable for delivery of the active ingredient to any suitable subject, and can be manufactured in a manner that is itself known, e.g., by means of conventional mixing, dissolving, granulating, emulsifying, encapsulating, entrapping or lyophilizing processes. Pharmacological compositions can be formulated in a conventional manner using one or more pharmacologically (e.g., physiologically or pharmaceutically) acceptable carriers, as well as optional auxiliaries that facilitate processing of the active compounds into preparations which can be used pharmaceutically. Proper formulation is dependent upon the route of administration chosen. Thus, for injection, the active ingredient can be formulated in aqueous solutions. For transmucosal administration, penetrants appropriate to the barrier to be permeated are used in the formulation. Such penetrants are generally known in the art.

For oral administration, the active ingredient can be combined with carriers suitable for incorporation into tablets, pills, capsules, liquids, gels, syrups, slurries, suspensions and the like. The active ingredient can be formulated for parenteral administration by injection, such as by bolus injection or continuous infusion. Such compositions can take such forms as suspensions, solutions or emulsions in oily or aqueous vehicles, and can contain formulatory agents such as suspending, stabilizing and/or dispersing agents. Other pharmacological excipients are known in the art.

Optionally, a CD70 agonist can be contained within or conjugated with a heterologous protein, hydrocarbon or lipid, whether for in vitro or in vivo administration. Co-administration can be such that the therapeutic agent is administered before, at substantially the same time as, or after the protein, hydrocarbon, or lipid. Other delivery systems can include time-release, delayed release or sustained release delivery systems. Such systems can avoid repeated administrations of the compositions of the invention described above, increasing convenience to the subject and the physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono-, di-, and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the at least one CD70 agonist, or polynucleotide encoding the agonist is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Use of a long-term sustained release implant may be particularly suitable for treatment of chronic conditions, such as scleroderma. Long-term release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. These systems have been described for use with oligodeoxynucleotides (see U.S. Pat. No. 6,218,371). For use in vivo, nucleic acids and peptides are preferably relatively resistant to degradation (such as via endo- and exo-nucleases). Thus, modifications, such as the inclusion of a C-terminal amide, can be used.

The therapeutically effective amount of a CD70 agonist, such as a sCD27 polypeptide, an antibody that specifically binds CD70, an antigen binding fragment of the antibody, a small molecule, or polynucleotide a CD70 agonist, will be dependent on therapeutic agent that is utilized, the subject being treated, the severity and type of the affliction, and the manner of administration. For example, a therapeutically effective amount of a polynucleotide encoding the peptide can vary from about 0.01 μg per kilogram (kg) body weight to about 1 g per kg body weight, such as about 1 μg to about 5 mg per kg body weight, or about 5 μg to about 1 mg per kg body weight. The exact dose is readily determined by one of skill in the art based on the potency of the specific compound the age, weight, sex and physiological condition of the subject.

With regard to the administration of nucleic acids, one approach to administration of nucleic acids is direct treatment with plasmid DNA, such as with a mammalian expression plasmid. As described above, the nucleotide sequence encoding a sCD27 polypeptide, an antibody that specifically binds CD70, and/or an antigen binding fragment of the antibody, can be placed under the control of a promoter to increase expression of the molecule.

When a viral vector is utilized for administration in vivo, it is desirable to provide the recipient with a dosage of each recombinant virus in the composition in the range of from about 10⁵ to about 10¹⁰ plaque forming units/mg mammal, although a lower or higher dose can be administered. The composition of recombinant viral vectors can be introduced into a mammal either prior to any evidence of fibrosis, or to mediate regression of fibrosis in a subject with a particular condition. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Alternatively, the recombinant viral vector or combination of recombinant viral vectors may be administered locally by direct injection into a fibrotic lesion in a pharmaceutically acceptable carrier. Generally, the quantity of recombinant viral vector, carrying the nucleic acid sequence of one or more CD70 agonists, such as a sCD27 polypeptide to be administered is based on the titer of virus particles. An exemplary range of the immunogen to be administered is 10⁵ to 10¹⁰ virus particles per mammal, such as a human.

In one specific, non-limiting example, a pharmaceutical composition for intravenous administration would include about 0.1 μg to 10 mg of a CD70 agonist, such as a sCD27 polypeptide, an antibody that specifically binds CD70, an antigen binding fragment of the antibody, or a small molecule, per patient per day. Dosages from 0.1 up to about 100 mg per patient per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceutical Sciences, 19th Ed., Mack Publishing Company, Easton, Pa., 1995.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

In a further method, an additional agent is administered. In one example, this administration is sequential. In other examples, the additional agent is administered simultaneously with the CD70 agonist.

For the treatment of scleroderma, examples of additional agents that can be used include nifedipine, amlodipine, diltiazem, felodipine, or nicardipine. An investigational drug Gleevec, is also used for the treatment of scleroderma. Gleevec or other tyrosine kinase inhibitors can be used with the CD70 agonists disclosed herein, such as, but not limited to, sCD27. Patients with lung involvement of scleroderma benefit from oxygen therapy; the CD70 agonists disclosed herein, such as, but not limited to, sCD27, can be administered with this therapy.

For the treatment of fibrosis of the skin and scleroderma, additional agents of use are d-penicillamine, colchicine, Relaxin, steroids, and cyclosporine. CD70 agonists, such as, but not limited to, sCD27, also can be used in combination with immunosuppressive agents. Additionally, the CD70 agonists, such as, but not limited to, sCD27, can be used with rituximab, pirfenidone, nintedanib, methotrexate, cyclophosphamide, azathioprine, mycophenolate, glitazones, endothelin receptor antagonists, or Fulvestrant (ICI-182,780).

The disclosure is illustrated by the following non-limiting Examples.

### EXAMPLES

### Example 1

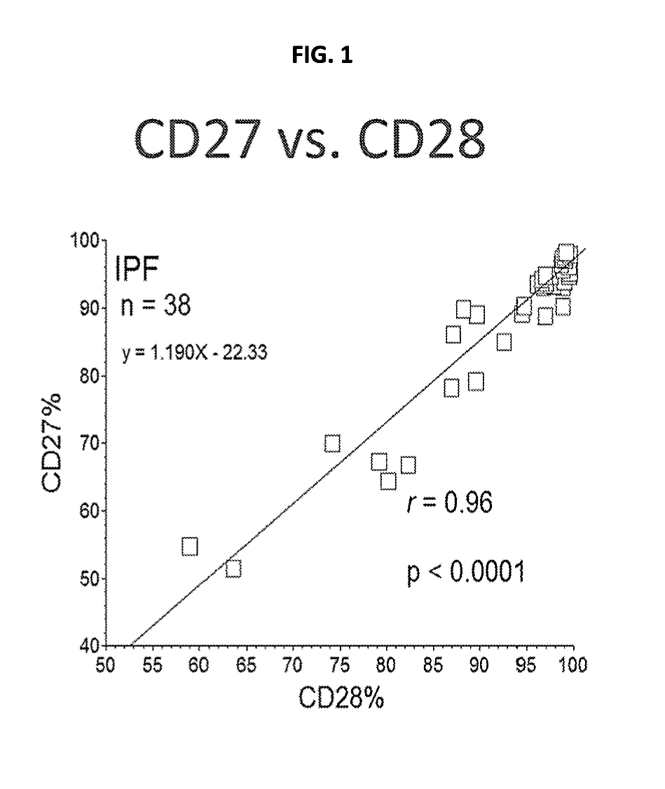

In idiopathic pulmonary fibrosis, T cells express CD27 and CD28 as shown in FIG. 1.

These data show a high correlation between expression of CD28 and CD27 on cell surfaces of circulating T-cells (CD4 T-cells shown here). Similar stringent associations between CD28 and CD27 have been seen in multiple disease populations (IPF, chronic obstructive pulmonary disease, lung allograft rejection). CD28 (and CD27) downregulation is a consequence of repetitive T-cell stimulation and replication induced by antigens (or auto- or allo-antigens) and is a specific and disease-associated marker of chronic immunological disease (Studer SM, et al., Am J Resp Critical Care Med. 2008; 178:765-773. PMID: 18617642; Gilani SR et al., Plos One 2010; 5:e8959 (doi:10.1371); Herazo-Maya J, et al., Sci Translat Med 2013; 5:205ra136. PMID: 24089408). As T-cells are stimulated and divide, the daughter progeny progressive alter functions, including their ability to mediate homeostatic regulation of fibrosis by CD27 (since there is less of it). The data were generated by flow cytometry of circulating CD4 T-cells, with methodologies previously detailed (Studer SM, et al. Am J Resp Critical Care Med. 2008; 178:765-773. PMID: 18617642; Gilani SR et al. Plos One 2010; 5:e8959 (doi:10.1371)).

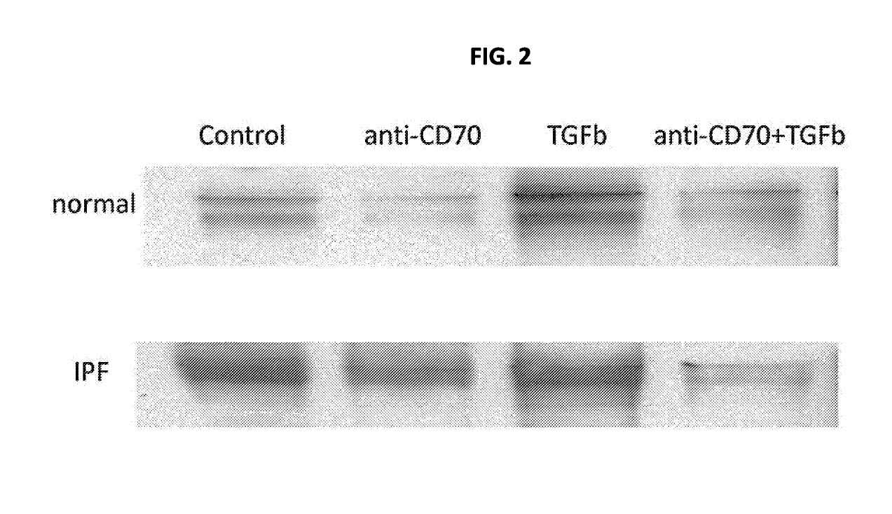

Collagen production by human fibroblasts is decreased by treatment with anti-CD70 (or CD27), and this modulates the stimulatory effect of transforming growth factor (TGF)-β as shown in the digital image of FIG. 2. Primary pulmonary fibroblasts from normal and IPF lung explants were cultured in DMEM supplemented with 10% heated-inactivated FCS, L-glutamine, hepes, and penicillin and streptomycin. IPF and normal fibroblasts at passage 3-5 were seeded at 1 or 2×10⁵/well in a final volume of 1 ml in 24-well plates and incubated for indicated time periods at 37° C. in a humidified atmosphere containing 7% CO₂. Cells were confluent for 24 hours, then stimulated with mouse anti-human CD70 mAb (Ancell, Cat. 222-020) at 2 μg/ml, mouse IgG control Ab (AbD Serotec, MCA928) at the same concentration (control for anti-CD70 mAb), recombinant human IgG CD27 Fc chimera (R&D, Cat. 110-HG) (2 μg/ml), recombinant human IgG Fc (R&D) (control for sCD27) in the presence or absence of TGF-β1 (R&D, Cat. 240-B-002) (2 ng/ml) for 24 hours. Extracellular matrix proteins were detected by Western blots using standard methods. In brief, the cells were suspended in 100 μL of lysis buffer (Cell Signaling, Cat. 9803) per well and incubated on ice for 30 minutes. Sample loading buffer was then added and the mixture boiled for 5 minutes. Cell lysates were resolved by 4-12% SDS-PAGE and electro-blotted to polyvinylidene difluoride membrane. Blots were blocked in 5% milk and incubated with primary antibodies, which included collagen-al (1/200, Santa Cruz, sc-8783). After incubation with HRP-conjugated secondary antibody, bound IgGs were detected using a SUPERSIGNAL™ West Pico chemiluminescent substrate kit (Thermo Scientific) following the manufacturer's instructions. Treatment with sCD27 had near identical effects on decreased production of collagen, and antagonism of TGF-β effects.

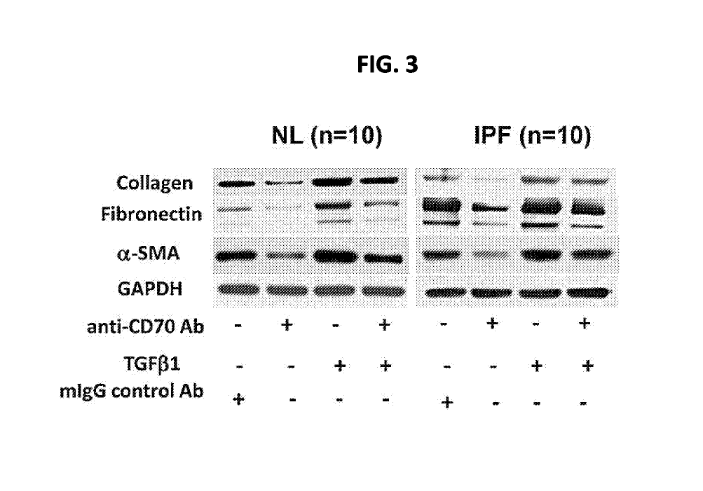

The CD70 antibody inhibits the production of various extracellular matrix (ECM) proteins by human fibroblasts as shown in the digital image of FIG. 3. Methods are nearly identical to those described in FIG. 2, with the addition of testing with a-SMA (1/200, Abcam, Cat. Ab5694), fibronectin (1/200, sc-8422) and anti-GAPDH (protein loading control). Treatment with sCD27 had nearly identical effects on decreased production of these ECM proteins, and antagonism of TGF-β effects.

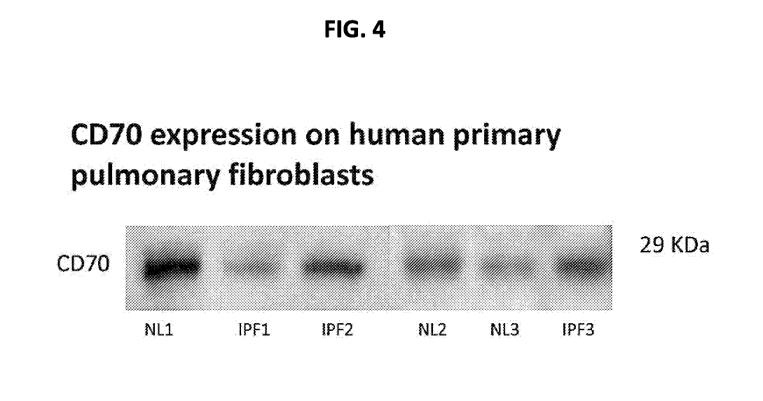

Western blotting was used to document the expression of CD70 in human pulmonary fibroblasts as shown in the digital image of FIG. 4. Whole cell lysates from primary pulmonary fibroblasts were extracted by RIPA lysate buffer. Sixty micrograms of protein from each sample was subjected to a SDA-PAGE for western-blot, using anti-CD70 antibody (Ancell) and anti-mouse IgG, as described above. Other studies using flow cytometry show that at least some of this CD70 is on fibroblast cell surfaces and is accessible to anti-CD70 antibodies, and sCD27 and other CD70 agonists.

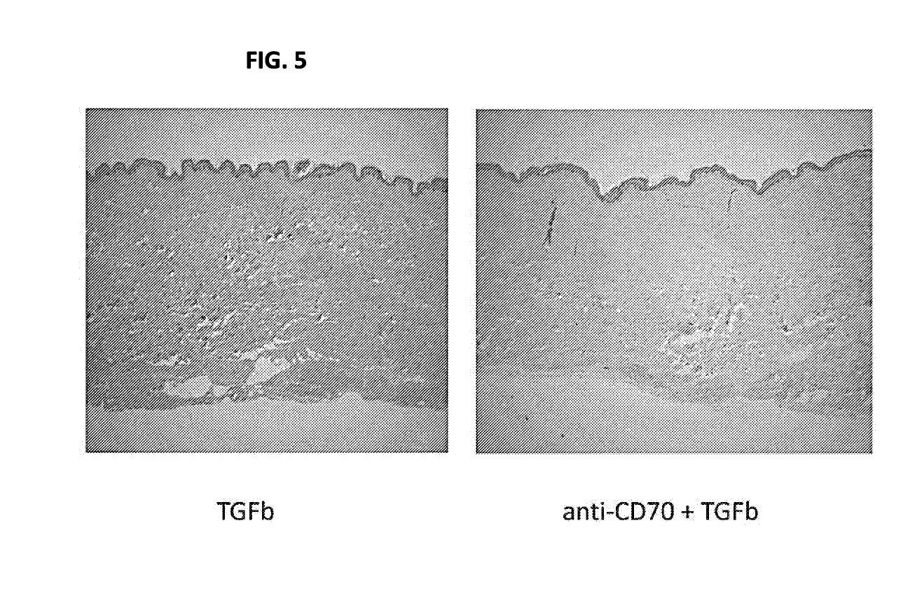

Human abdominal skin was treated with TGF-β (left panel) or TGF-β (right panel) and an anti-CD70 antibody as shown in the digital image of FIG. 5. Skin thickness was reduced when the skin was treated with the anti-CD70 antibody. Human abdominal skin harvested during surgical procedures was sectioned into 1.5 mm sections and cultured in an air-fluid interface, as described in Yasuoka H, et al. Human skin culture was used as an ex vivo model for assessing the fibrotic effects of insulin-like growth factor binding proteins. Open Rheum J 2008; 2:17-22. Intradermal injections of TGF-β, the anti-CD70 antibody, and sCD27 (from sources previously described), in the amounts denoted in the figure, were made 1 week prior to harvest, fixing, and H and E staining.

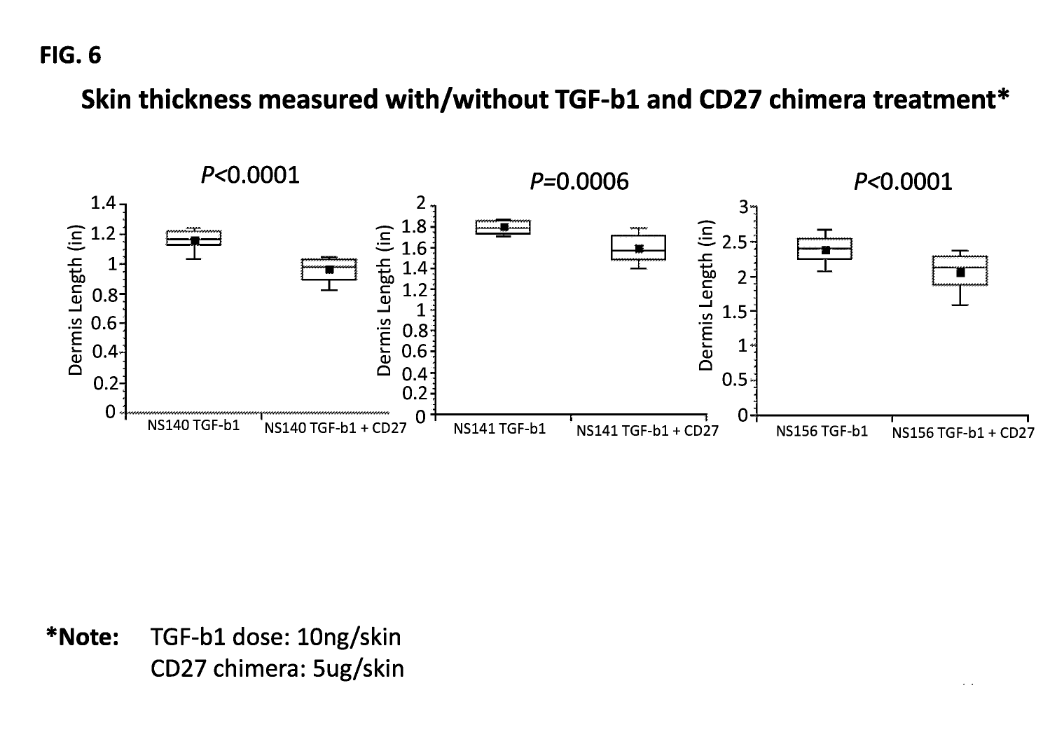

In response to stimulation with TGF-β, skin thickness decreased by sCD27 as shown in the set of graphs of FIG. 6. Skin thickness at 5 randomly selected cross sectional areas in each specimen was determined by directly measuring images (see FIG. 5) and was averaged and compiled in FIG. 6.

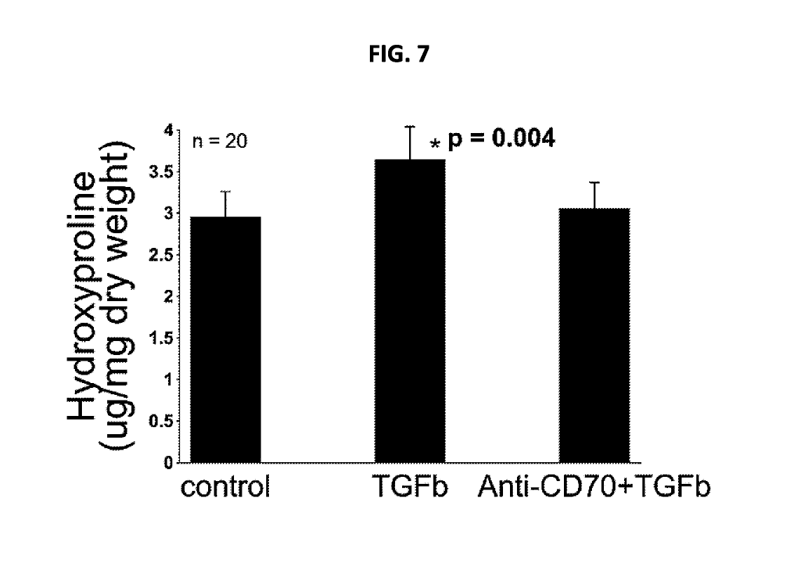

Punch biopsies (3 mm diameter) were made of human foreskins that had been obtained during circumcisions. Skin sections were grown in conditions described for abdominal skin. Sections were bathed in media supplemented with various permutations of TGF-β (10 ng/ml), anti-CD70 antibody (5 μg/ml), and appropriate controls. Following culture for 6 days, hydroxyproline in the specimens was measured using the Sigma kit, and adjusted to dry weight as shown in the bar graph of FIG. 7. TGF-β increased hydroxproline concentrations in these specimens, and this was blocked by the anit-CD70 antibody (P value by Friedman statistic).

### Example 2

### In Vivo Experiments

Mouse fibroblasts do not express CD70, nor alter their production of extracellular matrix proteins with CD70 engagements (by anti-CD70 antibodies or a soluble CD27 fusion protein). Accordingly, in vivo experiments require human (or higher primate) fibroblast targets. The effectiveness of anti-CD70 (or sCD27) treatments using ex vivo human abdominal skin and foreskins has been demonstrated (see Example 1). Additional models are:

1. Human-murine chimera: Human pulmonary fibroblasts (isolated from lung explants) are injected into immunodeficient mice (Pierce et al. Am J Pathol 170: 1152-1164, 2007, incorporated herein by reference). They colonize the mouse lungs and induce pulmonary fibrosis. This chimeric model is used to test effects of anti-CD70 and CD27-fusion protein, with and without treatment with TGFb-1, as an additional in vivo model.

2. Human skin xenograft model: Human skin (abdominal skin excised during therapeutic surgical procedures or foreskins removed during circumcisions) is grafted onto lateral abdomens of immunodeficient mice. These animals are treated as described above for Human-murine chimeras.

3. Non-human primate mode (NHPM): Interventions with anti-CD70 and sCD27 fusion protein employ, as a preclinical trial, a NHPM as discussed in Wilder, et al., Journal of Inflammation 2013, 10(Suppl 1):P39, http://www.journal-inflammation.com/content/10/S1/P39, as available on July 3, 2015, incorporated herein by reference.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of treating a subject with fibrosis and/or reducing the thickness of the skin, comprising administering to the subject a therapeutically effective amount of a CD70 agonist, or a nucleic acid encoding a CD70 agonist, thereby treating the subject with fibrosis and/or reducing the thickness of the skin.

2. The method of claim 1, wherein the subject has fibrosis of the skin, and wherein the method reduces skin thickness in the subject.

3. The method of claim 1, wherein the subject has scleroderma, a keloid, or a hypertrophic scar.

4. The method of claim 1, wherein the subject has morphea, Graft-Versus-Host Disease, or sub-epithelial fibrosis.

5. The method of claim 1, wherein the subject has pulmonary fibrosis, and the method reduces fibrosis in the lungs of the subject.

6. The method of claim 5, wherein the pulmonary fibrosis is idiopathic pulmonary fibrosis.

7. The method of claim 5, wherein the method reduces a pathologic rate of decline of a pulmonary function parameter by at least five percent.

8. The method of claim 7, wherein the pulmonary functional parameter is vital capacity (VC), forced expiratory volume (FEV), forced vital capacity (FVC), forced vital capacity percent (FVC %) predicted, functional residual capacity (FRC), inspiratory capacity (IC), total lung capacity (TLC), expiratory reserve volume (ERV), tidal volume (TV), or maximum voluntary ventilation (MVV).

9. The method of claim 1, wherein the agent decreases extra-cellular matrix production.

10. The method of claim 1, wherein the agent is soluble CD27.

11. The method of claim 10, wherein the soluble CD27 comprises an amino acid sequence at least 95% identical to SEQ ID NO: 1, or 95% identical to amino acids 1-189 of SEQ ID NO: 1, and wherein the soluble CD27 binds to CD70.

12. The method of claim 11, wherein the soluble CD27 comprises amino acids 1-189 of SEQ ID NO: 1, and wherein the soluble CD27 binds to CD70.

13. The method of claim 1, wherein the agent is a monoclonal antibody that specifically binds CD70 or an antigen binding fragment of the monoclonal antibody.

14. The method of claim 13, wherein the monoclonal antibody is a human antibody.

15. The method of claim 1, wherein the agent is CDX-1127 or ARGX-110, or an antigen binding fragment thereof.

16. The method of claim 1, further comprising administering to the subject a therapeutically effective amount of another therapeutic agent.

17. The method of claim 1, wherein the CD70 agonist decreases extra-cellular matrix production.

18. The method of claim 1, wherein the subject is human.

19. The method of claim 2, further comprising measuring the thickness of the skin.

20. The method of claim 2, wherein the CD70 agonist is administered locally to the skin.

21. The method of claim 5, wherein the CD70 antagonist is administered locally to the lungs.

22. The method of claim 5, further comprising measuring pulmonary function of the subject.

23-28. (canceled)


SEQUENCE LISTING (2 sequences)

SEQ ID NO: 1 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 2 - DNA, 783 bp, Homo sapiens
atggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga